(19) United States
(12) Patent Application Publication
(10) Pub. No.: US 2006/0251686 A1
(43) Pub. Date: Nov. 9, 2006

(54) Make-up processes and processes for application of a skin care product, and devices used in the implementation of such processes

(76) Inventor: Jean-Louis H. Gueret, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/538,017

(22) PCT Filed: Dec. 4, 2003
(86) PCT No.: PCT/EP03/13698

§ 371 Date: Jun. 16, 2006

Related U.S. Application Data
(60) Provisional application No. 60/438,988, filed on Jan. 10, 2003.

(30) Foreign Application Priority Data
Dec. 13, 2002 (FR) 02/15823
Apr. 16, 2003 (FR) 03/04774

(51) Int. Cl. A61K 8/31; H05B 6/80

(52) U.S. Cl. 424/401; 219/725

(57) ABSTRACT

The present invention relates to a process for the application of a cosmetic product, including a skin care product, excluding depilatory waxes, this product being contained in a packaging device, the process including the following stages: placing the packaging device in a microwave oven, raising the temperature of the product by exposing it to microwave radiation inside an oven, applying the product by means of an applicator. The invention also relates to packaging devices for cosmetic products capable of being heated in a microwave oven.

The present invention relates to make-up processes and processes for application of a skin care product, and also to the devices used in the implementation of such processes.

It has been proposed to apply cosmetic products after having raised their temperature. Thus, French patent application FR 2 376 401 proposes that shampoos be heated to a temperature slightly higher than that of the human body in order to achieve improved effectiveness. To this end, quantities of shampoo are placed in an apparatus incorporating heating elements. U.S. Pat. No. 5,775,344 describes a packaging and applicator device for mascara incorporating a heating element integral with the container.

International application WO 00/43286 describes a device incorporating components which, when mixed, produce an exothermic reaction enabling the temperature of a cosmetic composition to be raised.

Document DE-43,12,278 describes the preparation of a cosmetic composition by pre-heating the concentrated phase in a microwave oven before adding the water to be mixed with this concentrated phase in order to obtain said cosmetic composition.

The object of the invention, according to a first aspect inter alia, is a process for the application of a cosmetic product, including a body care product, excluding depilatory waxes, this product being contained in a packaging device, the process including the following steps:

place the packaging device in a microwave oven,

raise the temperature of the product by exposing it to microwave radiation inside an oven,

apply the product by means of an applicator.

The term “microwave oven” means conventional ovens as used in other applications to heat food and including a chamber in which an item to be heated can be exposed to electromagnetic energy.

Compared with heating by exposure to a source of infrared radiation, the use of a microwave oven enables a rapid increase in temperature to be achieved irrespective of the heat-conducting characteristics of the walls of the container holding the product.

The product can thus be brought quickly and easily to the desired temperature without having to incorporate a heating element into the packaging device. The temperature at the core of the product mass is raised rapidly. The temperature rise of the product progressively heats the walls of the container which are in contact with it. The product is also heated in a uniform manner by this type of radiation. Heating of the product can also be used to render the product homogeneous, repeatedly if necessary. There is no separation of the chemical phases of the product by virtue of the fact that the temperature rise is uniform.

Heating of the product can be used for example to facilitate spreading or retention on an area to be coated, for example the eyelashes, hair, skin or mucous membranes, or to improve the waving of hair. In particular, heating the product can improve its dermatological properties and/or improve its lengthening, curling, shining, matifying, softening, and or anti-wrinkle effects. Heating can be effective in the context of aromatherapy, to promote the effects of the aromas and the essential oils applied. Hot application of a cosmetic product can also simulate a sauna or hammam effect on the skin in the case of topical application. It can promote the penetration of an active ingredient of the product into the skin, mucous membranes or keratin fibres, and exert a local action on blood circulation, for example.

For example, the product may produce at least two types of effects depending on the temperature at which it is used. Advantageously, the product possesses properties which enable it to be applied either hot or cold, in particular at ambient temperature. This can enable the user to adapt the properties of a product to best suit the type of application, make-up or body care desired. For example, hot application of the product allows a thinner layer to be applied in order to obtain lighter coverage of make-up, and application of the same product at ambient temperature can be used to achieve a matifying effect.

The rheological properties of the product may be modified by temperature, in particular viscosity, surface tension, structural composition, and thixotropic properties, where appropriate. Heating can then facilitate take-up of the product. Then heating also facilitate impregnation of applicator, notably porous or foam applicators.

For example, in the case of extemporaneous preparations including at least two components, for example two liquids or a powder and a liquid, for example extemporaneous hair care preparations, heating may accelerate mixing of the components and/or the dissolution of one component in another.

Heating can also be used to re-liquefy product which has dried on the walls of the container, and to cause it to flow into the bottom of the container to facilitate emptying.

Heating by microwaves provides a means of bacterial purification, or pasteurisation, of the product held in the container. This pasteurisation can be carried out several times during the life of the product. This heating means that product formulations containing fewer preservatives can be envisaged.

Preferably, the product contains water or any other substance which absorbs the radiation emitted by microwave ovens and which heats up on exposure to microwaves.

An additional advantage associated with the use of microwaves to raise the temperature of the product lies in the possibility of incorporating thermal insulation permeable to microwaves into the packaging device, enabling heat to be retained by the product during use, and/or providing a holding surface cooler than the temperature of the product contained in the receptacle.

The product can be heated in the microwave oven in a manner such that its temperature is between 30° C. and 80° C., for example.

The duration for which the product is exposed to microwave radiation can be between 1 and 60 seconds for example, preferably between 2 and 50 seconds, or between 3 and 25 seconds, being for example close to 5 seconds. Heating of the product can be divided into several heating stages with pauses in between. These pauses may allow the user to check the temperature reached by the product.

This duration can depend in particular on the power of the oven, the nature of the container, the initial temperature and final temperature to be reached, and on the quantity and nature of the product. The device can include a table informing the user of the required heating time in relation to the power of the oven, for example.

The process can be put into effect once or several times, depending on whether or not the device is designed for single use.

The device may include a container having a shape making it possible to increase the exposure to microwave radiation. The container may in particular include a mark indicating to the user the position in which the container should preferentially be placed in the microwave oven. The container may for example be arranged so that the height of the product is less in a heating position than in a normal usage position of the container. The height of the product is measured vertically between the lower level of the product and its upper level. The heating position may for example be a recumbent position to allow for the horizontal distribution of a microwave field in the oven chamber.

Alternatively, the heating position can be a raised position, achieved for example by means of a stand for the packaging device, so that the device is exposed to maximum radiation by being placed in a central zone of the oven space. The stand is placed for example on the oven bottom and by cooperation with the device enables the container to be raised relative to the oven bottom. The height to which the device is placed may be function of heating temperature to reach, and/or volume of the product to be heated. For example, the stand raise the device from 1 to 5 centimetres relative to the oven bottom.

In the case of containers fitted with an applicator tip, in particular made of foam, or a wiper, these containers are preferably arranged so that the applicator tip and/or wiper are respectively heated by contact with the product heated at its core under the effect of the microwave radiation. The closed container is placed “upside down” inside the microwave oven.

The container can include a wall arranged to allow it to be placed stably in a recumbent position, for example a wall having a prismatic transverse cross-section offering at least one flat surface rather than a circular transverse cross-section.

The packaging device may include a sign informing the user of the possibility of placing it in a microwave oven. This sign may for example consist of a form of words such as “microwave advanced technology”.

A further object of the invention, according to another of its aspects, independently or in conjunction with the foregoing, is a packaging device incorporating an applicator and container holding a cosmetic product, including a skin care product, excluding depilatory waxes, the device being capable of being heated in a microwave oven. For example, the applicator is first detached from the container before the product is exposed to microwave radiation.

For example, the applicator can be kept out of the oven when the product is being raised to temperature, in particular when the applicator is incompatible with heating in a microwave oven. On the other hand, the applicator can also be placed in the microwave oven, in cases where this is made possible, with a view to exposing it to such radiation, for example in order to raise the surface temperature of the applicator, and in particular at its application surface.

Preferably the device includes an indicator designed to provide information on the temperature of the product.

The packaging device or an outer packaging for example indicate the possibility of heating the packaging device in a microwave oven. An indicator of this kind can inform the user about the temperature of the product when it is removed from the microwave oven, to enable the product to be used properly at the desired temperature, and in particular to safeguard the user against being burned.

As the device is designed to allow the product to be heated in a microwave oven, it is advantageously devoid of metal, for example metal components or metallic or electrically conductive coatings, preferably being made only of materials compatible with use in a microwave oven. After heating in the microwave oven, the outside temperature of the container is generally lower than the inside temperature of the product.

The indicator used may in particular serve to warn the user when the temperature of the product is higher than at least one pre-defined value. This indicator can be integral with the container holding the product. In cases where the device includes a closure element for the container, the indicator can be integral with this closure element. The device may also include an applicator and the indicator can then be integral with this applicator, and the indicator can thus be directly in contact with the product during the time that the device is placed in the microwave oven. In the case where the device includes a container with fitted base, the indicator can be integral with this base or can be held by it on the container. The base is for example snapped or welded onto the container. Where appropriate, the indicator can also be presented by the stand.

The temperature-sensitive indicator can be arranged in a variety of ways on the device. The indicator can be detachable or permanently fixed on the packaging device. Where the indicator is detachable, it takes the form for example of an elastomer ring incorporating thermochromic pigments capable of being slid onto and retained on an outer periphery of the container. The indicator can for example include at least one flexible support fixed, for example by gluing or welding, on the device. The indicator can for example take the form of a pad or an adhesive label affixed to a wall of the device, for example a wall of the container or a closure element of the container.

The indicator can also be made by printing or screen printing on the device using an ink incorporating a thermochromic pigment. The indicator can also be formed by incorporating a thermochromic pigment into the material of at least part of the device, for example the material of at least part of the container, at least part of a closure element of the container, at least part of a fitted base where applicable, or at least part of the applicator, for example at least part of an applicator element or rod connecting the applicator element to a handling element, which may serve where applicable to close the container.

The device incorporating such thermochromic pigments may for example be obtained by bi-injection. In this case, a viewing panel incorporating said pigments can be made in a wall of the container.

The indicator may include any material which changes its appearance with temperature, for example of cholesteric liquid crystals, possibly encapsulated, and preferably a material compatible with placement of the device in a microwave oven. For examples of materials which change their appearance according to temperature reference may be made for example to patent application EP 1 191 317 A1 or U.S. Pat. No. 5,786,578.

The temperature sensitive indicator may for example change appearance, in particular its colour, with temperature, by changing from one colour to another when a pre-defined transition temperature is reached. The indicator can also present a transparency effect which is temperature-dependent, becoming transparent or opaque when a certain temperature is reached. Preferably, the indicator changes its appearance in a reversible manner with temperature, i.e. it reverts to its initial appearance when the device returns to its initial temperature. The indicator may also present a colour saturation effect which is a function of temperature.

The indicator is chosen in relation to its transition temperature and in relation to the thickness of the wall to which it is applied to take account, where applicable, of the thermal inertia, heat transfer coefficient, thermal resistance, and heat absorption coefficient of this wall. In this case, an indicator is chosen that changes state at a temperature lower than the temperature to which it is desired to heat the product, to allow for the temperature difference which may exist between the product and the indicator when the latter is not directly in contact with the product but is separated from it by the wall of the container.

Two kinds of indications may be required. Firstly, the user may wish to determine that the temperature reached by the product is not dangerous, i.e. that it is lower than a first temperature Tₘₐₓ or T₁, for example in the order of 50° C. Secondly, the user may also wish to know if the temperature of the product has been raised effectively by the heating process, and that it is in fact higher than a second temperature Tₕₒₜ or T₂, for example in the order of 30° C., to ensure that product is applied “hot”. When the temperature is between Tₕₒₜ and Tₘₐₓ, the correct conditions are established for “hot” use of the product.

In a first embodiment, provision is made for the use of two separate indicators on the device, each of these indicators having a different transition temperature. A first indicator having a transition temperature to show when the temperature T₁ has been exceeded, and a second indicator having a transition temperature to show when the temperature T₂ has been passed. The chosen values for the transition temperatures are for example several ° C. below T₁ and T₂ by reason of the thermal resistance of the walls between the indicator and the product.

In a second embodiment, provision is made for one temperature indicator placed on a wall of the device having at least two different thicknesses. Thus, by varying the thermal resistance of this wall, linked to its thickness, it is possible to have the same indicator change over in a first region, and remain unchanged in a second region. When the indicator has changed colour only in the thin-wall region, and the thick-wall region of the same indicator remains unchanged, the user can then pick up the container and apply the product. Preferably the regions where the wall is thicker serve as the grasping surfaces as they are in fact cooler. In this case, to ensure that the indicator is reliable, the thickness of the wall of the container at the point where the indicator is attached must be precisely controlled.

To obtain such a container having a variable wall thickness, provision is made for example for horizontal, vertical or helical ribs projecting beyond an external outline of the container. As a variant, the container presents a circular inner outline on a transverse cross-section, whereas its external outline may be of any shape except that of a similar circular form of which the centre is superimposed on that of the inner outline. For example, the external outline, on this same transverse cross-section, can take the form of an excentric circle, ovoid, triangle, rectangle, or any type of polygon.

The device can also include an applicator for the product held in the container. This applicator may or may not be exposed to microwave radiation. Where the applicator cannot be heated in this way, it is placed in a holder during the time that the container is exposed to microwave radiation. For example, this holder is designed to be attached to the container in a storage position during periods when the container is not being heated.

This applicator is preferably integral with a plug enabling the container to be closed. The applicator can be made by moulding in a single piece with the plug, or can be fitted thereon and held in place by over-moulding or gluing.

The device can include at least one safety valve to avoid excessive pressure build-up inside the device if the latter is inadvertently exposed to microwave radiation for too long a period.

The device may also include an anti-splash element to reduce the risk of product being expelled when the device is opened, under the effect of excess pressure created by heating the product, caused for example by the expansion of air. An element of this kind may or may not be actuated by the user before opening. Thus, in one embodiment, the anti-splash element includes a part on which the user can press to balance the pressure between the inside and the outside of the container, before opening the container.

As a variant, the anti-splash element automatically performs its function when the container is opened or when the applicator is withdrawn. This anti-splash element can also in particular serve to wipe the applicator. In a further variant, it is the applicator itself that can be used to reduce the risk of expulsion of product on opening, for example by forming a barrier against possible splashing of the product.

The device can also include a flow-reducing element serving for example to reduce the risk of loss of product in case the container is accidentally overturned, in particular if the fluidity of the product has greatly increased with temperature.

A further object of the invention, independently or in combination with the foregoing, is a method for promoting the sale of a packaging device containing a cosmetic product, including a body care product, excluding depilatory waxes, which makes known the possibility of placing the device in a microwave oven to raise the temperature of the product in order for example to modify the properties of the latter, in particular its rheology. This product is for example a body care or make-up product such as a foundation or a mascara.

A further object of the invention, independently or in combination with the foregoing, is a method for promoting the sale of a packaging and application device containing a cosmetic product, which makes known the possibility of obtaining two different make-up effects according to whether the product is used hot or cold.

Promotion of the product can be effected through any channel of communication. This may be done by a vendor, directly at the point of sale, or via radio or television, in particular in the context of advertising commercials. It may also be done through press publications or by means of any other document, in particular for advertising purposes. It may also be done through a computer network or mobile telephone network. It may also be done on the packaging device, other packaging media or in accompanying instructions.

A further object of the invention, according to another of its aspects, independently or in combination with the foregoing, is a packaging device for a cosmetic product including:

a container having a wall made at least partially in a first material,

a thermal insulator defining at least partially the external surface of the container, this thermal insulator being for example made in a second material having a thermal conductivity lower than that of the first material,

a cosmetic product, including a body care product, held in the container.

A further object of the invention is the use of such a device in a process including the following steps:

raising the temperature of the product preferably by placing it in a microwave oven,

applying the product by means of the applicator.

A further object of the invention, independently or in combination with the foregoing, is a device including:

a container,

a cosmetic product, including a body care product, held in the container,

an applicator equipped with an applicator element.

a wiper element arranged to wipe the applicator element as it leaves the container, the applicator and wiper element being designed to allow the air to escape on opening the device in case of excess pressure in the container.

A further object of the invention is the use of such a device in a process including the following steps:

raising the temperature of the product preferably by placing it in a microwave oven,

applying the product.

A further object of the invention is the utilisation of a device including:

a container,

a cosmetic product held in the container,

an applicator incorporating at least one part made in a material having a heat capacity enabling it to store heat,

in a process including the following steps:

raising the temperature of the product preferably by placing it in a microwave oven,

applying the product.

The desired heat capacity can be obtained for example by using a ceramic or a plastic material incorporating a high percentage, for example greater than or equal to 60% by weight, of a charge, for example mineral, in particular metallic or a ceramic or a porous structure capable of taking up the product within its depth, the latter then storing the heat.

The invention will be better understood from the following detailed description of non-limitative embodiments of the invention, and by reference to the attached drawings in which:

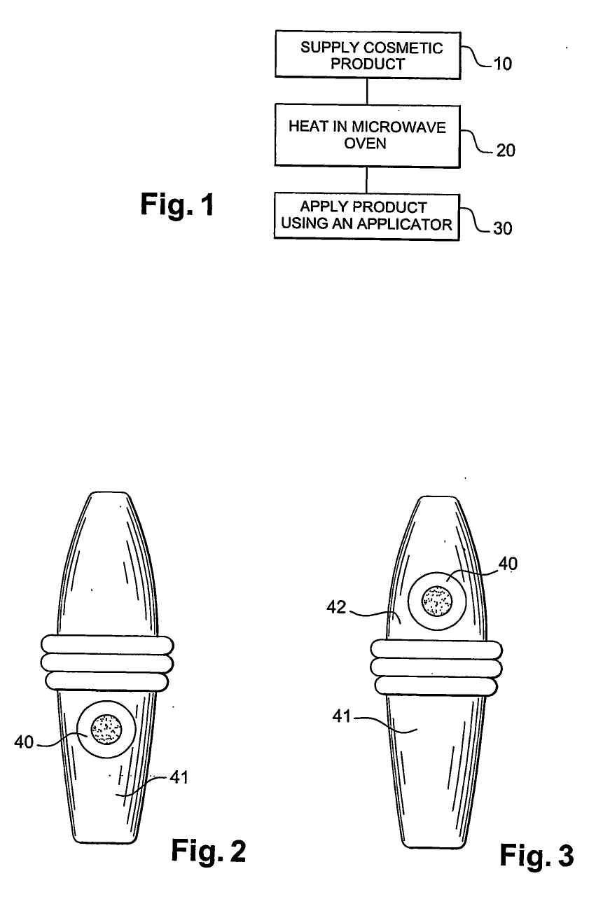

FIG. 1 is a block diagram showing the stages of an example of the process according to the invention,

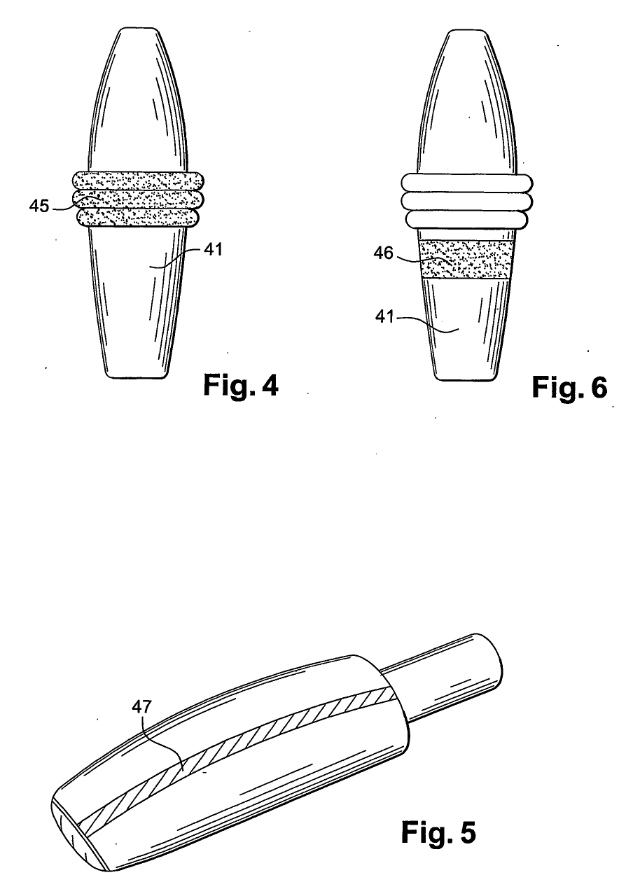

FIGS. 2 to 10 illustrate different possible embodiments, inter alia, of the temperature sensitive indicator,

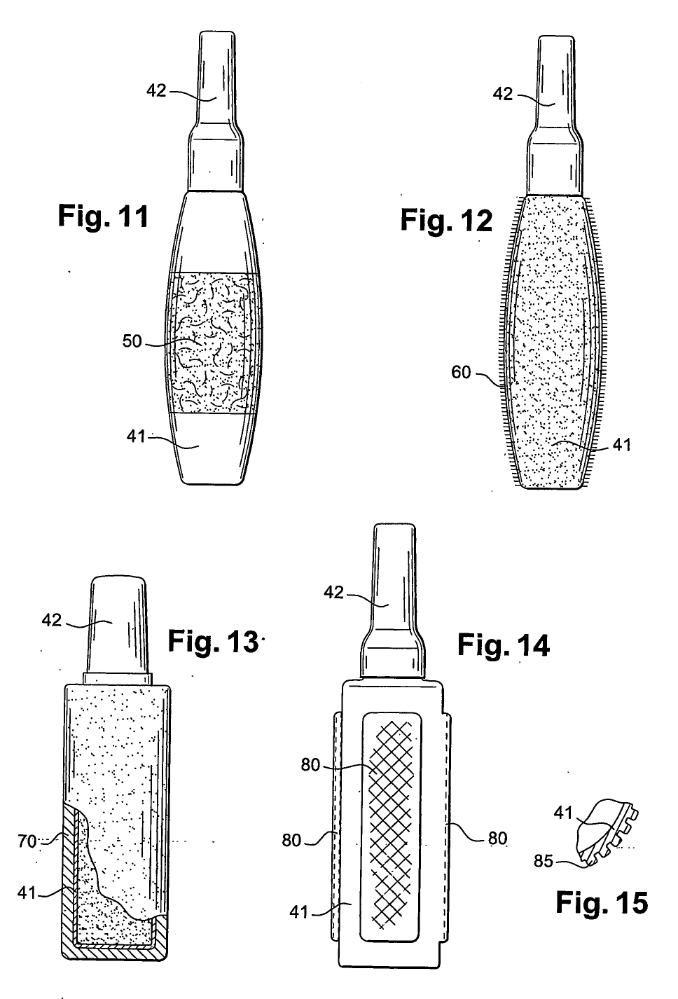

FIGS. 11 to 15 show different possible embodiments of thermal insulation on the container,

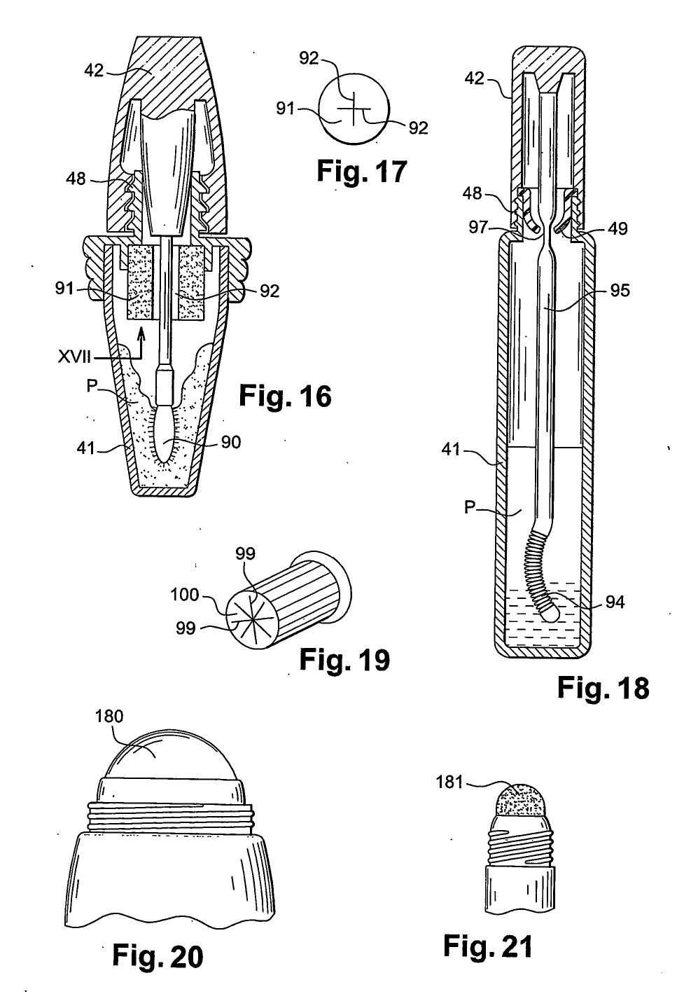

FIG. 16 is an embodiment of an anti-splash element allowing excess pressure to escape,

FIG. 17 shows a bottom view in isolation of the anti-splash element on arrow XVII in FIG. 16,

FIGS. 18 and 19 show other examples of means enabling excess pressure to escape,

FIG. 20 shows a partial view in elevation of an example of a rotary applicator,

FIG. 21 shows a partial view in elevation of an example of a device including a foam applicator integral with the container,

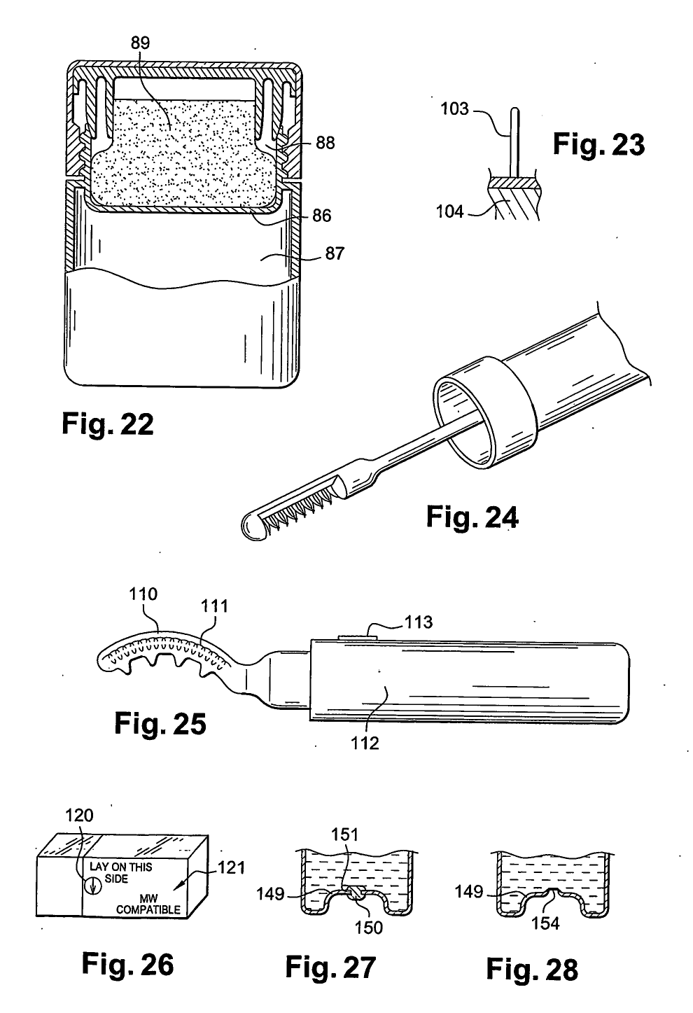

FIG. 22 is a schematic and partial view in longitudinal section of an example of a device including an applicator element integral with a closure element for the container,

FIG. 23 shows an over-moulded bristle in isolation,

FIG. 24 shows an example of an applicator made in a material having a high heat capacity,

FIG. 25 is a schematic illustration of a device intended to curl the eyelashes and incorporating a heating element,

FIG. 26 illustrates an embodiment of a container having a shape offering a stable recumbent position;

FIGS. 27 and 28 are partial schematic illustrations of containers equipped with active safety means in case of excess pressure in the container;

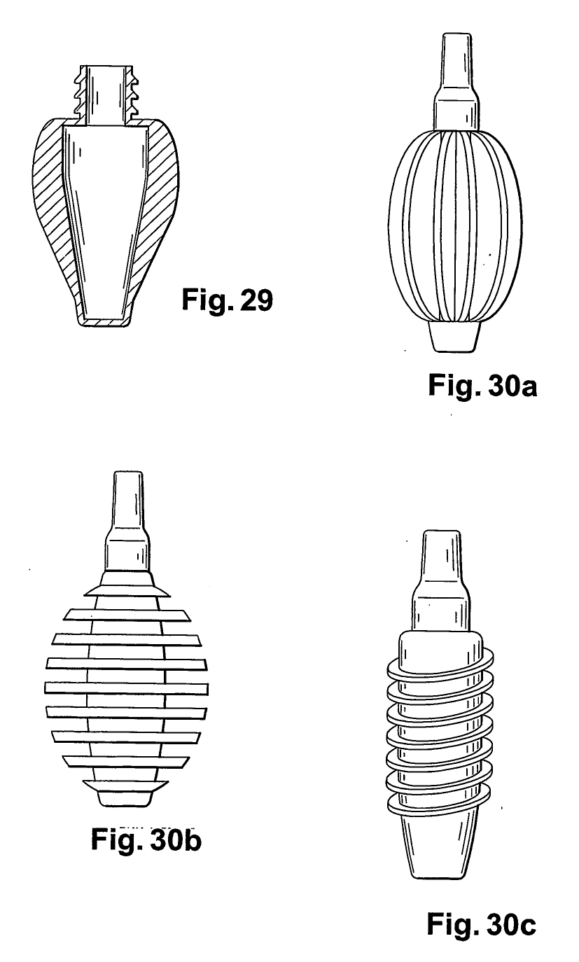

FIG. 29 is a sectional longitudinal view of a device according to the invention wherein the receptacle includes a wall of variable lengthwise thickness;

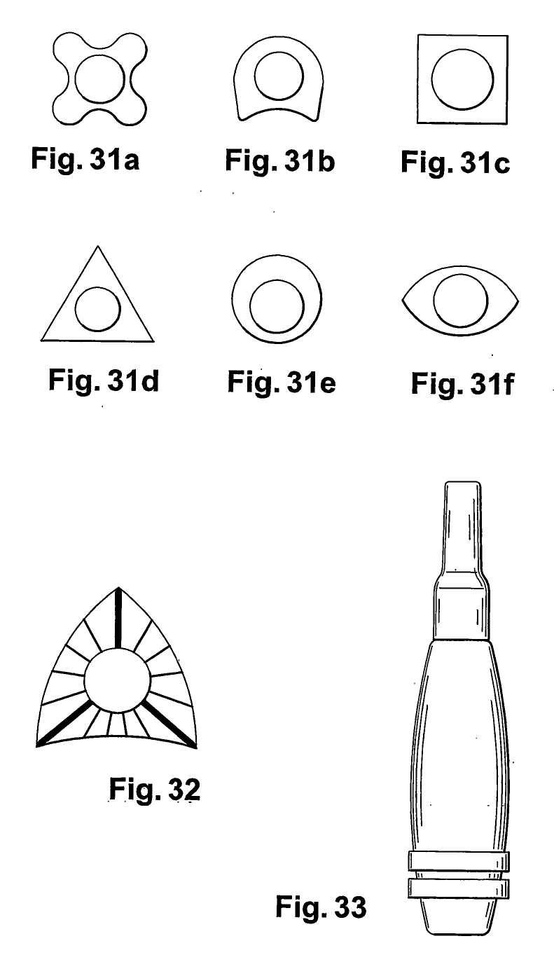

FIGS. 30a, 30b and 30c are partial schematic illustrations of containers for the device according to the invention provided with lengthwise, transverse and helical ribs respectively on their external outline;

FIGS. 31a to 31f show transverse sectional views of containers for the device according to the invention, wherein the wall thickness of the container can be variable on the external outline in this sectional plane;

FIG. 32 shows a transverse sectional view of a container for a device according to the invention incorporating lengthwise ribs of variable width, these ribs extending radially relative to an inner circular outline of the receptacle;

FIG. 33 shows a device according to the invention wherein the indicator is presented in the form of a ring slid onto and retained on a substantially cylindrical exterior outline of the receptacle,

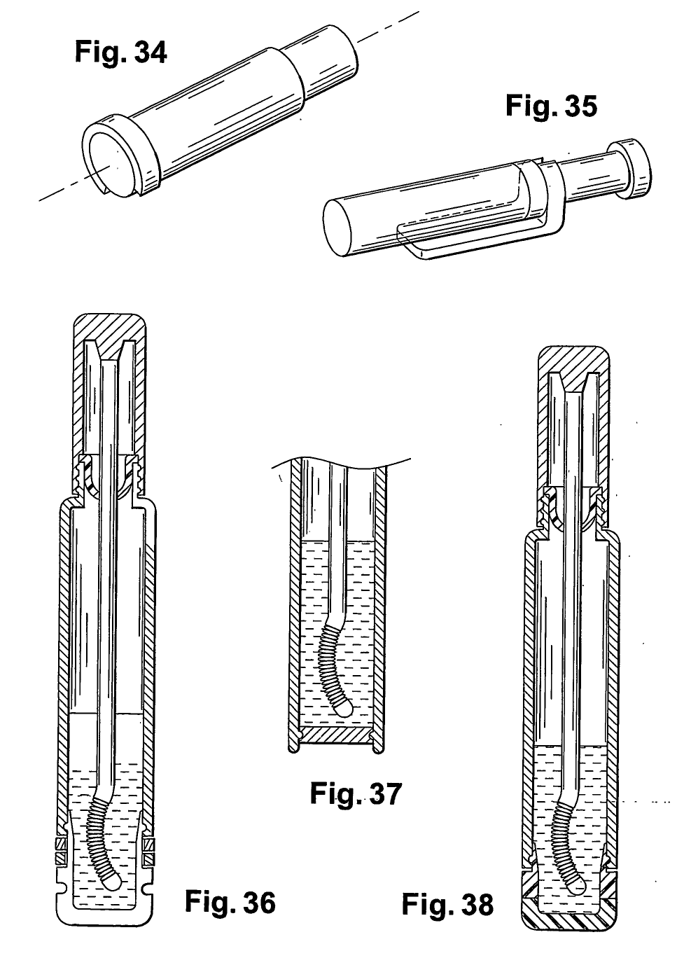

FIG. 34 shows a device according to the invention wherein the indicator is presented in the form of a U encircling at least partially the external outline of the device, the free ends of this U bearing against a plane along which the device is laid. Thus the U indicator also serves to immobilise the device, which in this instance is substantially cylindrical in shape, in a recumbent position relative to the surface;

FIG. 35 illustrates a device according to the invention wherein the indicator is presented in the form of a clip including a first portion designed to be clipped around the external outline of the device, and a second portion designed to bear against a surface parallel to which the device is laid. This clip type indicator thus serves to immobilise the device, which in this instance is substantially cylindrical in shape, in a recumbent position relative to the surface, and also serves to raise the device relative to this surface;

FIG. 36 shows a longitudinal sectional view of a device according to the invention incorporating a fitted base and an applicator mounted in a cap to close the receptacle. The fitted base is mounted on the container in a leaktight manner, by means of a snap-on attachment, but also so as to hold at least one indicator, in this instance two toroidal indicators held between the base and the receptacle.

FIG. 37 shows a longitudinal sectional view of a receptacle for a device according to the invention, this receptacle including a screw-in fitted base, wherein the base is made of a material incorporating thermochromic pigments capable of serving as an indicator;

FIG. 38 shows a longitudinal sectional view of a device according to the invention incorporating a fitted base and an applicator mounted in a cap to close the receptacle. The fitted base is mounted on the container in a leaktight manner, by screwing, the base being bi-injected so as to present at least one portion forming the temperature indicator, preferably visible from the external surface of the base;

FIG. 39 shows a view of a device according to the invention incorporating a viewing panel through which the product contained in the receptacle can be seen; and

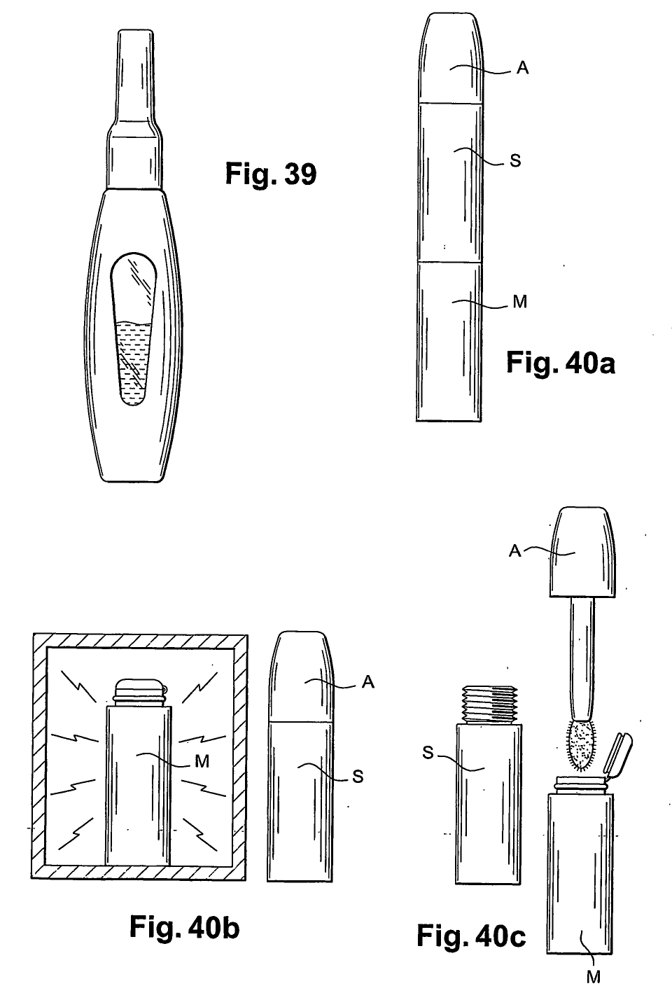

FIGS. 40a, 40b and 40c show three successive stages in the process according to the invention wherein a container M intended to be heated in a microwave oven is first detached from another holder S which receives the applicator A during the time that the receptacle is being heated, this applicator A being equally capable of mounting either on the container M or the holder S. Once the container M has been heated in the microwave oven, FIG. 40b, the applicator A is detached from the holder S in order to take up a quantity of heated product from the container M. Even if dried product remains on the applicator A, this dried product will be heated by contact with the heated product while introducing the applicator within the receptacle. When not in use, as shown in FIG. 40a, the container M can be made integral with the holder S in which the applicator A is stowed.

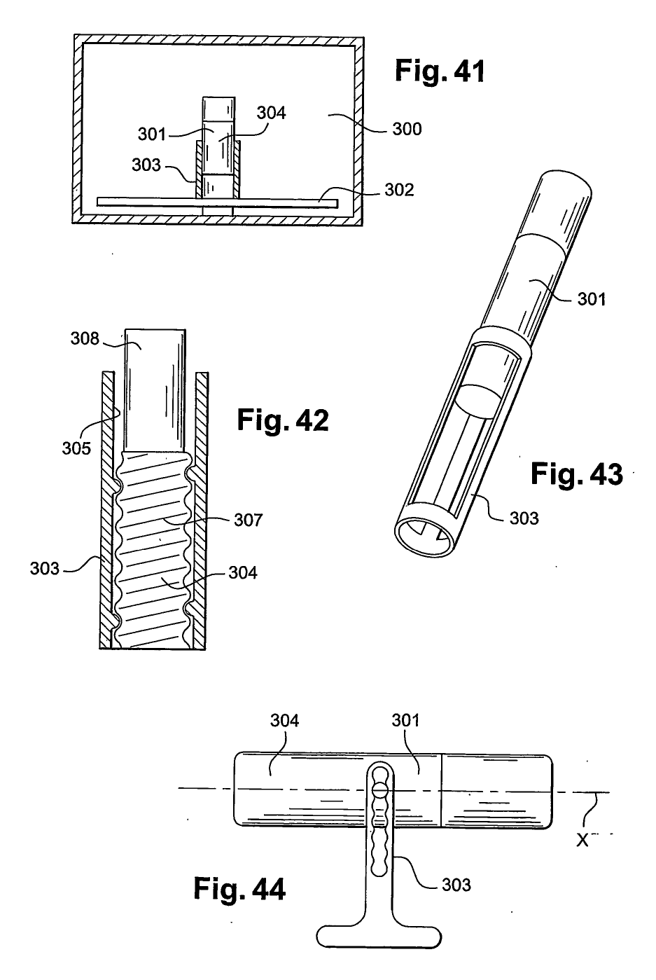

FIG. 41 shows a schematic view of a chamber 300 of a microwave oven inside which is placed a device 301 according to the invention, this device containing a cosmetic product and being capable of being heated in an oven of this kind. For example, the chamber 300 is equipped with a turntable 302 on which is placed the device 301. Preferably, the device 301 is placed at the centre of the turntable 302. In the embodiment shown, the device 301 is raised relative to the turntable 302 by means of a stand 303. The stand 303 allows the container 304 of this device 301 to be placed higher than the surface formed by the turntable 302. Thus, the container 304 can be placed in a central region of the chamber 300, in which the microwave radiation is more intense.

FIG. 42: according to a first particular embodiment of the invention, the stand 303 takes the form of a sleeve incorporating a stud 306 on its inner wall 305. For example, it includes several such studs 306. This sleeve engages with a screw thread 307 provided in this instance around the outer periphery of the container 304. For example, a means of closure 308 of the device 301 projects beyond the sleeve irrespective of the extent to which the stud 306 is engaged with the screw thread 307. Thus the user can easily set the device 301 in a high position relative to the stand 303 when they are placed in the oven. Alternatively, the stud can be provided on the outer periphery of the container 304 while the inner wall 305 of the sleeve offers a counterpart screw thread.

FIG. 43: according to a second particular embodiment of the invention, the stand 303 takes the form of a perforated sleeve which can be moved along the container 304.

FIG. 44: according to another particular embodiment of the invention, the stand 303 takes the form of a pivoting means about an axis orthogonal to a principal axis of elongation X of the device 301. In particular, the pivot is made at the level of the container 304. For example, the stand 303 can be used to place the device in an elongated and raised position relative to the surface of the turntable 302. The axis X is then parallel to this surface. As a variant, the pivoting means can hold the axis X orthogonal to the surface of the turntable 302, while at the same time allowing the receptacle to be raised relative to this surface.

The block diagram in FIG. 1 illustrates different stages in a process that can be used to raise the temperature of a cosmetic product according to a first aspect of the invention.

This process includes a first stage 10 involving the supply of the cosmetic product in a packaging device, possibly fitted with an applicator. The cosmetic product can be supplied via any mode of sale, in particular by sale in a shop or by mail order, or through a beauty establishment or hairdressing salon, for example.

The process then includes a stage 20 which involves heating the product in a microwave oven. The packaging device is suitable for placement in the microwave oven and preferably does not include any metallic or electrically conductive element liable to be damaged during exposure to microwave radiation or to damage the oven used.

The emissive power of the microwave radiation and the duration of exposure of the product to this radiation are selected in relation to the target temperature to be reached and to the contents of the packaging device. Preferably, the power of the microwave oven is selected so that the time for which the product is exposed to the microwave radiation is relatively short, for example less than 20 seconds, in particular in the order of several seconds.

The packaging device is then taken out of the microwave oven and stage 30 is carried out, involving application of the product. This application can be accomplished for example by means of an applicator which can be detachably mounted on the container holding the product when the latter has been placed in the microwave oven or separate from the packaging device. Application can also be accomplished using a finger, for example.

The product may for example be a lip colour, a mascara, a foundation or a skin care product, without this list being limitative.

At ambient temperature, i.e. at 20° C., the product can exhibit properties, in particular rheological properties, which allow application at this temperature, in particular that of being liquid at ambient temperature, as well as other properties which also allow application when the temperature is higher, for example above 30° C.

At the end of the heating period in the microwave oven, the temperature of the product inside the packaging device may exceed 50° C., or 70° C. Advantageously, the packaging device is equipped with a temperature sensitive indicator, in particular an indicator which changes its appearance, for example its colour, with temperature. This indicator can for example take the form of a label or pad 40 glued onto the side wall 41 of the container, as illustrated in FIG. 2, or glued to a closure element 42 of the container, as illustrated in FIG. 3. The closure element 42 may possibly constitute the grasping element of an applicator of which the application element is held inside the container when the latter is closed.

The temperature indicator can also be made by blending a thermochromic pigment with the material intended for example to form the upper part 45 of the container. In the example in FIG. 5, the temperature indicator takes the form of a strip 47 made for example by bi-injection or co-extrusion with the wall of the container body, for example in a material which changes colour according to temperature. This strip can serve as a viewing panel if it is made in a material that is transparent at least at certain temperatures.

Screen printing may also be used to affix, for example on the side wall 41 of the container, a strip 46 as illustrated in FIG. 6 or any other motif using an ink incorporating a thermochromic pigment.

In the examples shown in FIGS. 2 to 6, the indicator is visible from the outside. The scope of the invention is not exceeded when the indicator is not visible from the outside when the container is closed but becomes visible at the time of application or when the user performs a particular action, for example by withdrawing the indicator from the container.

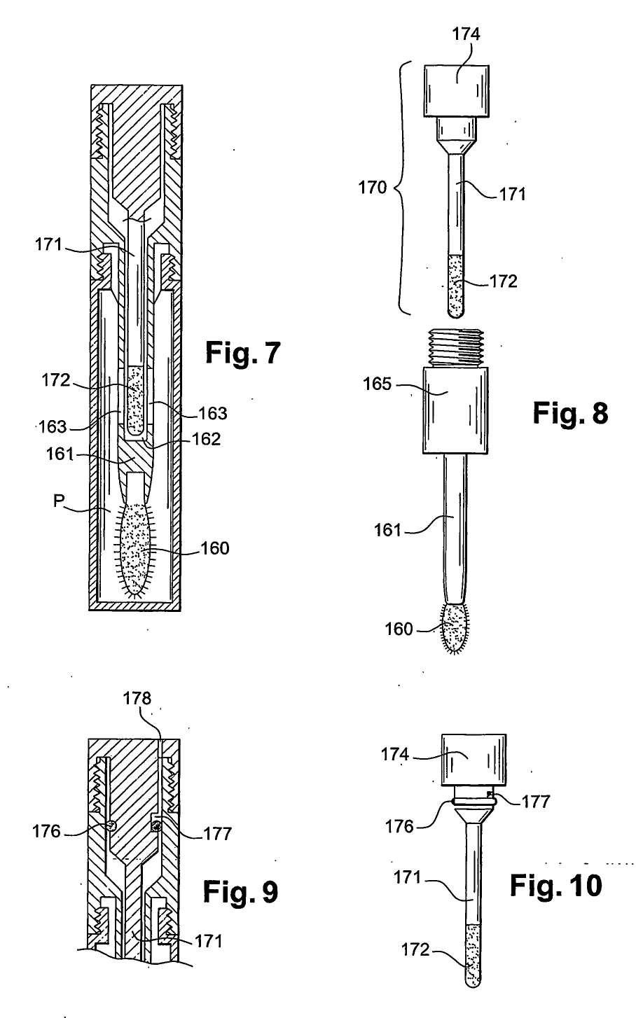

FIGS. 7 and 8 illustrate a device having a container holding the product P and an applicator incorporating an applicator element 160 attached to the end of a rod 161 including a seating 162 into which a temperature indicator 170 can be inserted. The latter includes a rod 171 of which the end 172 changes colour for example according to temperature. Openings 163, where appropriate, may be made in the rod 161 if required to allow the product P to come into contact with the temperature indicator 170. In the example considered, the rod 171 is connected at its upper extremity to a threaded part 174 which can be screwed onto the grasping element 165 of the applicator.

Where appropriate, as illustrated in FIGS. 9 and 10, the temperature indicator can be attached to the device in a manner such that it also serves as a safety valve in case of excess pressure in the container.

The rod 171 of the temperature indicator can for example have an O-ring seal 176 which in the absence of overpressure provides a leaktight closure. An indentation 177 is provided in the rod 171 and in case of overpressure the O-ring seal 176 deforms locally by pushing up into this indentation 177, which allows the air under pressure to escape.

An aperture 178 can be made, where appropriate, in the upper part 178 of the temperature indicator to provide an outlet for the air.

The temperature indicator can be made by any known means, in particular using any known thermochromic pigment or any other material which changes colour with temperature and preferably compatible with placement of the packaging device in a microwave oven to heat the product.

According to another aspect of the invention, the packaging device can include a thermal insulator 50, as illustrated in FIG. 11, which can cover for example a part of the side wall 41 of the container holding the product. This insulator 50 can for example take the form of a sleeve made of a material less thermally conductive than that used to make the side wall 41 of the container, this sleeve being fixed in an annular groove on the container. Such an insulator 50 allows the container to be grasped more comfortably when the latter is removed from the microwave oven and when the temperature of the product is in the region of 70° C. for example.

The insulator 50 can be made for example of a polyurethane or polyethylene foam. The insulator can also be made differently, for example in the form of a flock coating 60 covering at least part of the side wall 41 of the container, as illustrated in FIG. 12. The insulator can also be made in the form of a casing 70, as shown in FIG. 13, this casing 70 having for example the general shape of a glove finger allowing the container to be inserted. The insulator can also be made in the form of strips 80 or other elements attached, for example by gluing, to the side wall 41 of the container, as illustrated in FIG. 14. The container can also be made for example with fins 85, as illustrated in FIG. 15.

According to another aspect of the invention, the packaging device can be configured to allow the excess air pressure created inside the container by heating the product to escape when the container is opened, at the same time limiting the risk of splashing or leakage of the product.

When the device is also used to apply the product and includes, as illustrated in FIG. 16, an applicator including an applicator element 90 and a wiper element 91 to swipe the applicator element as it leaves the container, the wiper element 91 is advantageously designed so as to allow any overpressure present inside the container after heating the product P to escape gradually when the container is opened.

The wiper element 91 can for example take the form of a block of foam incorporating a passage for the applicator element 90, this passage having at least one slot and for example two slots 92 in the example illustrated in figure 17. These slots 92 allow air under pressure to escape when the closure element 42 is unscrewed sufficiently from the neck 48 of the container, thereby limiting the risk of splashing of the product.

It is seen in FIG. 17 that the applicator element can take the form of a flock applicator, this applicator for example including an elastomer or plastic body with its surface covered in a flock coating. The applicator need not include a flock covering. Thus the applicator can be made for example by moulding a plastic into a particular shape, in particular a shape enabling the eyelashes or eyebrows to be combed.

FIG. 18 illustrates an applicator element 94 forming a comb, the latter being fixed at one end of a rod 95 connected to a grasping element which also constitutes a closure element 42 of the container.

In the example considered, the wiper and anti-splash element takes the form of an elastomer component mounted in the neck 48 of the container and provided at its lower end with a wiping lip 49 defining an opening, for example circular.

The rod 95 can include a groove 97 enabling air under pressure to escape when the container is opened. This groove 97 may be annular or may extend over only part of the circumference of the rod.

The wiper element can also be made with a plurality of radial slots 99, as illustrated in FIG. 19, these slots defining between them sectors 100 capable of deforming under the pressure of the air inside the container thereby allowing it to escape. Various other means can also be used to reduce the risk of splashing of the product in case of overpressure in the container.

FIG. 20 partially illustrates a device in which the applicator includes a rotary applicator element such as a ball 180 for example. This ball 180 enables the product to be applied while at the same time reducing the risk of splashing of the product when the closure cap, not shown, is removed.

The rotary applicator element can be replaced, for example, by a non-rotary applicator element permanently fixed on the container, such as a foam applicator 181 as illustrated in FIG. 21. Provision can also be made for a perforated wall 86, for example between a space 87 in the container holding the product and an opening 88 through which the product is taken up, as illustrated in FIG. 22. Where appropriate, this wall 86 can serve as the seating for an applicator element 89.

Generally speaking, when microwave radiation is used to raise the temperature of the product P, the packaging device is devoid of metallic elements and the applicator is made without metal. Thus, where it is desired to use a brush, the latter can be made with bristles 103 over-moulded on a support 104, as illustrated in FIG. 23, the bristles and support being for example made of different thermoplastic materials.

According to another aspect of the invention, the applicator element can be made in such a way that it possesses a sufficiently high thermal inertia that the product present on the applicator element does not cool down too quickly. It is thus possible to make the applicator element, such as for example a comb as illustrated in FIG. 24, in a plastic material incorporating a large proportion of a mineral or other charge imparting to it an elevated heat capacity.

The applicator element can thus be made by moulding a thermoplastic or thermo-hardening material incorporating a charge of a compound such as bronze or an aluminium oxide. It is in particular possible to make the applicator element by moulding a mixture incorporating 60% by weight of aluminium oxide and the remainder of polyamide or polypropylene. By way of a further non-limitative example, the applicator element can also be made by moulding a mixture incorporating 40% by weight of polypropylene and 60% of a ceramic.

FIG. 25 shows a device used to curl the eyelashes, incorporating an element 110 with an elevated heat capacity, intended to be brought into contact with the eyelashes for the purpose of imparting a curl to them, incorporating a heating element 111, which is connected by an electrical circuit not shown to an electrical power source contained in a handle 112, and a switch 113 enabling the element 111 to be energised. This device can include a thermostat system enabling the current to be interrupted when the element 110 reaches a sufficient temperature. This applicator device is detached from the receptacle and is not exposed to microwave radiation. It is used in addition to heating of the product.

The fact that the thermal inertia of the element 110 is relatively large, by virtue of the use of a charged plastic for example, can make it possible to reduce the power consumption of the device. The thermal inertia of the applicator element can also be increased by using a material to make the applicator that is capable of becoming internally loaded with the product to be applied, for example a porous material such as a foam or an agglomerate.

In addition, the container can advantageously be made with a shape enabling it to be laid flat without rolling in the microwave oven, in order to derive most benefit from the distribution field of the microwaves in the oven. By way of example, FIG. 26 illustrates a container of square transverse cross-section, capable of being laid on one of its sides. It can be seen in this figure that the container can include, for example, a reference mark 120 indicating to the user the most suitable way of placing the container in the microwave oven. The container may also include a notice 121 indicating the possibility of placing the device in a microwave oven.

To allow the product and air to escape in the event that the device is accidentally left too long in the microwave oven while it is in operation, a safety valve can be provided on the container. By way of example, FIG. 27 shows a partial axial cross-section of a container in which the bottom 149 is provided with a closure 150 forming a safety valve. This closure 150 is formed for example by an elastomer plug fixed in a hole 151 in the bottom wall 149 and capable of being expelled in case of excess pressure in the container. As a variant, the safety valve can be made by a constriction 154 in the bottom wall, as illustrated in FIG. 28. In case of overpressure, the wall is able to breach at the constriction.

Clearly, the invention is not limited to the examples described above. In particular, different features of the various embodiments can be combined between each other.

Throughout the description, including the claims, the expression “including a” should be understood to be synonymous with “including at least one”, unless otherwise specified.

1-17. (canceled)

18. A method of applying a cosmetic product to a surface, the method comprising:
increasing temperature of a cosmetic product arranged on or in a carrier substantially dry to the touch, the carrier being capable of being applied to a surface, and the temperature of the cosmetic product being increased via an energy source external to the carrier; and
applying the product to the surface.

19. The method of claim 18, wherein the product comprises a skin care product or a hair care product.

20. The method of claim 18, wherein the increasing temperature comprises increasing the temperature of the cosmetic product via microwave radiation.

21. The method of claim 18, wherein the increase in the temperature of the cosmetic product enables activation of at least one constituent of the cosmetic product.

22. The method of claim 18, wherein the increase in the temperature of the cosmetic product initiates or promotes activity of at least one constituent of the cosmetic product.

23. The method of claim 22, wherein the increase in the temperature of the cosmetic product promotes release of the at least one constituent to the surface.

24. The method of claim 18, wherein the increase in the temperature of the cosmetic product enables liquefaction of the product.

25. The method of claim 18, wherein the carrier is in the form of at least one layer.

26. The method of claim 18, wherein the carrier is made of a material comprising at least one of: cellulose, foam, woven material, felt, non-woven material, and plastic material.

27. The method of claim 18, wherein the carrier is immersed in a liquid before being heated.

28. The method of claim 27, wherein the liquid comprises water.

29. The method of claim 18, wherein the increasing comprises increasing the temperature of the cosmetic product to a temperature between 25° C. and 100° C.

30. The method of claim 29, wherein the increasing comprises increasing the temperature of the cosmetic product to a temperature between 30° C. and 55° C.

31. The method of claim 20, wherein the cosmetic product is exposed to the microwave radiation for a time period between 1 second and 150 seconds.

32. The method of claim 31, wherein the cosmetic product is exposed to the microwave radiation for a time period between 2 seconds and 60 seconds.

33. The method of claim 32, wherein the cosmetic product is exposed to the microwave radiation for a time period between 3 seconds and 25 seconds.

34. The method of claim 18, wherein the carrier is on a grid above or inside the container, wherein the container contains a liquid vaporizable at a heating temperature.

35. The method of claim 34, wherein the height of the grid is adjustable relative to the level of liquid in the container.

36. The method of claim 34, further comprising a lid arranged above the grid, wherein the lid and the container define a substantially closed volume around the carrier.

37. The method of claim 18, wherein the carrier includes a temperature indicator.

38. The method of claim 37, wherein the indicator is configured to change its appearance as a function of the temperature.

39. The method of claim 38, wherein the indicator is configured to change its color as a function of the temperature.

40. The method of claim 37, wherein the indicator is configured to change its material state at a predetermined temperature.

41. The method of claim 37, wherein the indicator comprises an ink or a label comprising a thermochromic pigment.

42. The method of claim 37, wherein the indicator is placed on at least part of a surface of the carrier.

43. The method of claim 41, wherein the indicator is placed on at least part of the surface of the carrier by printing.

44. A method of applying a cosmetic product to a surface, the method comprising:
increasing temperature of a cosmetic product arranged on or in a carrier comprising two non-occlusive application surfaces, the carrier being capable of being applied to a surface and the temperature of the cosmetic product being increased via an energy source external to the carrier; and
applying the product to the surface.

45. The method of claim 44, wherein the cosmetic product comprises a skin care product or a hair care product.

46. The method of claim 44, wherein the increasing temperature comprises increasing the temperature of the cosmetic product via microwave radiation.

47. The method of claim 44, wherein the increase in the temperature of the product facilitates liquefaction of the cosmetic product.

48. A carrier associated with a cosmetic product, the carrier comprising:
a body comprising a surface,
wherein at least part of the surface of the body comprises an indicator configured to change its appearance in response to an increase in temperature,
wherein the carrier is substantially dry to the touch, and
wherein the carrier is capable of being heated in a microwave oven.

49. The carrier of claim 48, wherein the cosmetic product is on or inside the carrier.

50. The carrier of claim 49, wherein the cosmetic product comprises a skin care product or a hair care product.

51. The carrier of claim 49, wherein the indicator is configured to change its color in response to a temperature increase.

52. A carrier associated with a cosmetic product, the carrier comprising:
a body comprising
a first non-occlusive application surface, and
a second non-occlusive application surface,
wherein at least part of one of the first and second surfaces comprises an indicator configured to change its appearance in response to an increase in temperature,
wherein the carrier is substantially dry to the touch, and
wherein the carrier is capable of being heated in a microwave oven.

53. The carrier of claim 52, wherein the cosmetic product is on or inside the carrier.

54. The carrier of claim 53, wherein the product is a skin care product or a hair care product.
